(19) United States
(12) Patent Application Publication
(10) Pub. No.: US 2026/0182927 A1
(43) Pub. Date: Jul. 2, 2026

(54) METHOD AND SYSTEM FOR DETECTING PULMONARY FUNCTION ABNORMALITIES BASED ON AUDIO SIGNALS

(71) Applicant: FAR EASTONE TELECOMMUNICATIONS CO., LTD., Taipei City (TW)

(72) Inventors: SHIH-LUNG CHENG, Taipei City (TW); CHIEN-NAN LEE, Taipei City (TW); CHUN-CHIEH KUO, New Taipei City (TW); HUA-PEI CHIANG, Taipei City (TW); CHYI-DAR JANG, Taipei City (TW); TSUNG-JEN WANG, Taipei City (TW); CHE-YU LIAO, Taipei City (TW)

(21) Appl. No.: 19/196,938

(22) Filed: May 2, 2025

(30) Foreign Application Priority Data
Dec. 31, 2024 (TW) 113151695

(51) Int. Cl. A61B 5/00; A61B 7/00; G16H 10/60; G16H 50/20

(52) U.S. Cl. CPC A61B 5/7267; A61B 5/4803; A61B 5/7203; A61B 5/7221; A61B 7/003; G16H 10/60; G16H 50/20; A61B 2560/0223

(57) ABSTRACT

A method and a system for detecting pulmonary function abnormalities based on audio signals are disclosed. The method combines the audio features and the individual parameters of a patient and normalizes them to generate an individual predicted pulmonary function parameter. Real pulmonary function parameters and augmented pulmonary function parameters are used as training data to overcome a problem with insufficient training data. Combining noise filtering techniques with diverse model training strategies can efficiently extract audio features and optimize the model performance to implement technology for non-invasively and conveniently detecting pulmonary functions.

### BACKGROUND OF THE INVENTION

This application claims priority for the TW application No. 113151695 filed on 31 Dec. 2024, the content of which is incorporated by reference in its entirely.

### FIELD OF THE INVENTION

The present invention relates to a technology field for detecting pulmonary function abnormalities based on audio signals, particularly to an application that combines audio features with a machine learning model for predicting pulmonary function abnormalities.

### DESCRIPTION OF THE RELATED ART

In modern medicine, the detection of pulmonary function abnormalities is a critical step in diagnosing and managing respiratory diseases. However, existing detection techniques still face numerous challenges in practical applications. Traditional pulmonary function testing methods typically require patients to go to medical institutions for testing and have high patient cooperation requirement, such as deep inhalation and forced exhalation. However, for certain patient groups, such as the elderly, those with severe pulmonary impairment, or individuals unable to accurately follow test instructions, these methods can lead to measurement deviations or even the inability to generate valid results. Therefore, the convenience and accuracy of these methods still need to be improved.

Furthermore, with the rapid advancement of machine learning technology, pulmonary function detection models trained based on clinical data have become a significant research topic. However, existing models often encounter challenges related to insufficient training data. Collecting pulmonary function parameters and their corresponding audio features from patients requires lots of time and resources, frequently resulting in a scarcity of labeled samples. This issue is particularly pronounced in underrepresented populations, such as patients with moderate to severe conditions or those with rare diseases. The scarcity of samples constrains the effectiveness of model training, thereby impacting the model's generalization ability in real-world applications.

Additionally, current machine learning models are typically static models, meaning they cease to be updated once training is completed. However, as new patient data are continuously generated, these static models may struggle to adapt to dynamic variations in data distribution, leading to a gradual decline in performance. Moreover, existing technology lacks effective methods for incrementally learning new data, preventing models from dynamically integrating new data while retaining previously acquired knowledge, thereby limiting their long-term applicability.

In summary, existing technology exhibits significant shortcomings in the accuracy of detecting pulmonary function abnormalities, the effectiveness of data augmentation, model adaptability, and noise filtering performance. Therefore, improved technology is needed to address these challenges and meet the requirements of more efficient, accurate, and flexible clinical applications.

### SUMMARY OF THE INVENTION

The primary objective of the present invention is to provide a method for detecting pulmonary function abnormalities based on audio signals, which combines the audio features and the individual parameters of a patient and normalizes them to generate an individual predicted pulmonary function parameter. Real pulmonary function parameters and augmented pulmonary function parameters are used as training data to overcome a problem with insufficient training data. Combining noise filtering techniques with diverse model training strategies can efficiently extract audio features and optimize the performance of models to implement technology for non-invasively and conveniently detecting pulmonary functions.

In order to achieve the foregoing objective, a method for detecting pulmonary function abnormalities based on audio signals includes: receiving from at least one tested person a raw audio signal that includes the breathing and/or vocalizing audio of the tested person; processing the raw audio signal and performing noise filtering on the raw audio signal to generate a filtered audio signal; extracting at least one audio feature from the filtered audio signal, wherein the audio feature includes a spectrum feature and/or a vocalizing feature; inputting the audio feature into a machine learning model to generate a first predicted pulmonary function parameter; and normalizing the first predicted pulmonary function parameter and converting the first predicted pulmonary function parameter into a second predicted pulmonary function parameter based on a preset reference standard, wherein the reference standard provides a basis for normalization according to the sex, age, and height of the tested person.

The method further includes a training process. The training process includes: obtaining the real pulmonary function parameter of at least one patient; and either using the real pulmonary function parameter as the labeled audio sample set of the machine learning model or using the real pulmonary function parameter to calibrate or validate the second predicted pulmonary function parameter.

The method further includes: tuning the real pulmonary function parameter to generate at least one augmented pulmonary function parameter; and either using the augmented pulmonary function parameter as the augmented labeled audio sample set of the machine learning model or using the at least one augmented pulmonary function parameter to calibrate or validate the second predicted pulmonary function parameter.

The method further includes: either calculating a difference value between the labeled audio sample set and the augmented labeled audio sample set or calculating a ratio of the labeled audio sample set to the augmented labeled audio sample set; and updating the parameter weight of the machine learning model and using the difference value or the ratio as an error value to calibrate the predicted accuracy of the machine learning model for pulmonary function abnormalities.

The method further includes: performing a cross-validation process to divide the labeled audio sample set and the augmented labeled audio sample set into subsets and using each of the subsets as a validation set in turn while the remaining subsets are used as the training set to evaluate the predicted accuracy of the machine learning model for pulmonary function abnormalities; and selecting the machine learning model with an optimal model configuration based on average accuracy or errors measured by the cross-validation process.

The method further includes: using a trichotomy method to divide the labeled audio sample set into a training set, a validation set, and a test set and dividing the augmented labeled audio sample set into the training set and the validation set; combining the training sets divided from the labeled audio sample set and the augmented labeled audio sample set to train the machine learning model; tuning the hyperparameters or the network structure of the machine learning model based on the predicted results of the validation set divided from the labeled audio sample set and the augmented labeled audio sample set; and using the test set divided from the labeled audio sample set to validate the predicted accuracy of the machine learning model for pulmonary function abnormalities after tuning the hyperparameters or the network structure.

The step of tuning the real pulmonary function parameter includes: correcting at least one of the age, height, and weight of the at least one patient to obtain the physiological condition of a virtual patient; calculating the augmented pulmonary function parameter corresponding to the physiological condition, wherein the augmented pulmonary function parameter is derived by a clinical formula and/or a physiological model; and determining whether the augmented pulmonary function parameter falls within an acceptable clinical range: if yes, including the augmented pulmonary function parameter into the augmented labeled audio sample set; and if no, excluding the augmented pulmonary function parameter.

The method further includes: receiving the new real pulmonary function parameter of at least one new patient; including the new real pulmonary function parameter into an incremental labeled data set and including the incremental labeled data set update the labeled audio sample set; and finely tuning the machine learning model and using the incremental labeled data set to perform another training on the machine learning model.

The present invention provides a system for detecting pulmonary function abnormalities based on audio signals, which includes a receiving module and a processing module. The receiving module is configured to receive or extract from at least one tested person a raw audio signal that includes the breathing and/or vocalizing audio of the tested person. The processing module is coupled to the receiving module and configured to perform noise filtering on the raw audio signal to generate a filtered audio signal, extract at least one audio feature from the filtered audio signal, input the audio feature into a machine learning model to generate a first predicted pulmonary function parameter, and convert the first predicted pulmonary function parameter into a second predicted pulmonary function parameter based on a preset reference standard.

The system further includes a training module coupled to the processing module and configured to obtain a real pulmonary function parameter of at least one patient. The training module is configured to either use the real pulmonary function parameter as the labeled audio sample set of the machine learning model or use the real pulmonary function parameter to calibrate or validate the second predicted pulmonary function parameter.

The system further includes a data augmentation module coupled to the receiving module, the processing module, and the training module and configured to tune the real pulmonary function parameter to generate at least one augmented pulmonary function parameter. The data augmentation module is configured to either use the augmented pulmonary function parameter as the augmented labeled audio sample set of the machine learning model or use the at least one augmented pulmonary function parameter to calibrate or validate the second predicted pulmonary function parameter.

The processing module is configured to either calculate a difference value between the labeled audio sample set and the augmented labeled audio sample set or calculate the ratio of the labeled audio sample set to the augmented labeled audio sample set, update the parameter weight of the machine learning model, and use the difference value or the ratio as an error value to calibrate the predicted accuracy of the machine learning model for pulmonary function abnormalities.

The processing module is further configured to perform a cross-validation process to divide the labeled audio sample set and the augmented labeled audio sample set into subsets, use each of the subsets as a training set and a validation set in turn to evaluate the predicted accuracy of the machine learning model for pulmonary function abnormalities, and select the machine learning model with an optimal model configuration based on average accuracy or errors measured by the cross-validation process.

The processing module is further configured to divide the labeled audio sample set into a training set, a validation set, and a test set, divide the augmented labeled audio sample set into the training set and the validation set, combine the training sets that are divided from the labeled audio sample set and the augmented labeled audio sample set to train the machine learning model, and tune the hyperparameters or the network structure of the machine learning model based on the predicted results of the validation set that are divided from the labeled audio sample set and the augmented labeled audio sample set. The processing module uses the test set divided from the labeled audio sample set to validate the predicted accuracy of the machine learning model for pulmonary function abnormalities after tuning the hyperparameters or the network structure.

When tuning the real pulmonary function parameter, the data augmentation module corrects at least one of the age, height, and weight of the at least one patient to obtain the physiological condition of a virtual patient, calculates the augmented pulmonary function parameter corresponding to the physiological condition, and determines whether the augmented pulmonary function parameter falls within an acceptable clinical range. The augmented pulmonary function parameter is derived by a clinical formula and/or a physiological model. When the augmented pulmonary function parameter falls within the acceptable clinical range, the data augmentation module includes the augmented pulmonary function parameter into the augmented labeled audio sample set.

The training module is further configured to receive the new real pulmonary function parameter of at least one new patient, include the new real pulmonary function parameter into an incremental labeled data set, include the incremental labeled data set update the labeled audio sample set, finely tune the machine learning model, and use the incremental labeled data set to perform another training on the machine learning model.

As mentioned above, the noise filtering includes a spectrum subtraction, which selects voice-free segments from the raw audio signal as background noise, calculates the average spectrum of the background noise, and linearly subtracts the average spectrum from the spectrum of the raw audio signal.

The noise filtering includes voice activity detection that is determined based on a short-time energy threshold. Short-time energy is the average value of square values of amplitudes of the raw audio signal within a predetermined time window. The short-time energy threshold is a dynamic reference value calculated based on environmental background noise. The short-time energy threshold is used to determine a voice activity. The method for determining the voice activity includes: comparing the short-time energy with the short-time energy threshold in each time window, and when the short-time energy in consecutive time windows is greater than the short-time energy threshold, determining that the voice activity begins; when the short-time energy in consecutive time windows is less than the short-time energy threshold, determining that the voice activity ends; and extracting the voice activity in a time period as a valid voice segment and excluding background noise.

The first predicted pulmonary function parameter, the second predicted pulmonary function parameter, the real pulmonary function parameter, and the augmented pulmonary function parameter include a peak expiratory flow, a best peak expiratory flow, an asthma control test score, a forced expiratory volume in one second, a forced vital capacity, or a combination of these.

Compared to the prior art, the present invention provides the following advantages:
1. Significant Improvement in the Accuracy of Detecting Pulmonary Function Abnormalities: By combining audio features and individual patient parameters (e.g., sex, age, and height) to normalize data, the first predicted pulmonary function parameter is converted into an individualized (normalized) second predicted pulmonary function parameter. This method overcomes a problem with existing audio-based detection technology that fails to account for individual patient differences, thereby significantly enhancing detection accuracy and clinical applicability. The method also demonstrates stronger generalization abilities in diverse patient groups.
2. Generation of Augmented Pulmonary Function Parameters to Address a Scarcity of Training Data: By tuning and augmenting the real pulmonary function parameter, the augmented pulmonary function parameters that reflect diverse physiological conditions among patients are generated. Specifically, age, height, and weight are corrected. The clinical formula or the physiological model is used to evaluate the parameter and to ensure that the augmented data maintains clinical plausibility when simulating virtual patient conditions. These augmented pulmonary function parameters serve as additional training data (e.g., training sets or validation sets), significantly enhancing data diversity.
3. Enhancement of Long-Term Model Performance: The existing machine learning model uses fine-tuning techniques. The model is allowed to continuously receive real pulmonary function parameters from new patients and to integrate them into the incremental labeled data set of the model for fine-tuning. This dynamic update mechanism ensures that the model quickly adapts to the distributional variations of clinical data, thereby improving long-term accuracy in applications.
4. Diverse Model Training Strategies: The diverse model training strategies including a cross-validation method and a trichotomy method (i.e., divided into training set, validation set, and test set) are employed to effectively balance the model's learning effect for real and augmented pulmonary function parameters and to improve the model's accuracy and generalization abilities. These strategies ensure stable training under different data conditions and use the validation set to tune the hyperparameters. Moreover, by avoiding using the augmented pulmonary function parameters as the technical solution of the test set, the risk of excessively tuning the hyperparameters of the test set is avoided, thereby efficiently predicting pulmonary function abnormalities.
5. Efficient Noise Filtering and Extraction of Audio Features: By combining spectrum features and vocalizing features, the precision and comprehensiveness of extracting audio features are enhanced. These vocalizing features effectively capture the subtle variations of voice signals, particularly the stability and clarity of voices. Thus, voice features associated with pulmonary function abnormalities are more accurately identified. This method also improves the model's ability to analyze voice quality and potential health issues, leading to more precise detection for pulmonary function abnormalities.
6. Method for Non-invasively and Conveniently Detecting Pulmonary Function Abnormalities: By using audio signals to detect pulmonary function abnormalities, the present invention provides a non-invasive alternative particularly beneficial for patients who cannot cooperate with traditional tests, such as children, the elderly, or critically ill patients. This detection method greatly enhances the convenience of the test process while reducing the burden on healthcare resources.

Below, the embodiments are described in detail in cooperation with the drawings to make easily understood the technical contents, characteristics and accomplishments of the invention.

### BRIEF DESCRIPTION OF THE DRAWINGS

FIG. 1 is a flowchart of a method for detecting pulmonary function abnormalities based on audio signals according to a first embodiment of the present invention;

FIG. 2 is a flowchart of a method for detecting pulmonary function abnormalities based on audio signals according to a second embodiment of the present invention;

FIG. 3 is a diagram schematically illustrating a system for detecting pulmonary function abnormalities based on audio signals according to a first embodiment of the present invention; and

FIG. 4 is a diagram schematically illustrating a system for detecting pulmonary function abnormalities based on audio signals according to a second embodiment of the present invention.

### DETAILED DESCRIPTION OF THE INVENTION

Reference will now be made in detail to embodiments illustrated in the accompanying drawings. Wherever possible, the same reference numbers are used in the drawings and the description to refer to the same or like parts. In the drawings, the shape and thickness may be exaggerated for clarity and convenience. This description will be directed in particular to elements forming part of, or cooperating more directly with, methods and apparatus in accordance with the present disclosure. It is to be understood that elements not specifically shown or described may take various forms well known to those skilled in the art. Many alternatives and modifications will be apparent to those skilled in the art, once informed by the present disclosure.

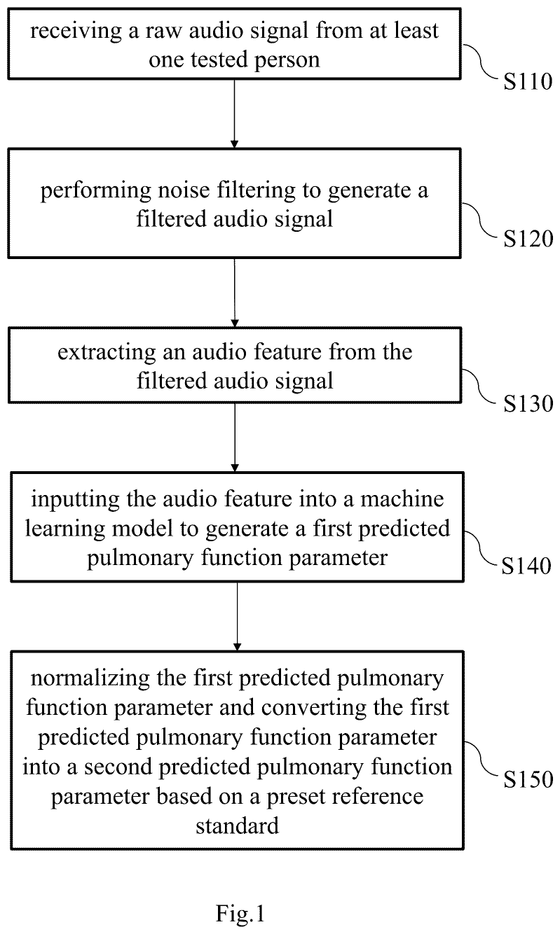

Please refer to FIG. 1. FIG. 1 is a flowchart of a method for detecting pulmonary function abnormalities based on audio signals according to a first embodiment of the present invention. The flowchart includes Steps S110˜S150.

In Step S110, a raw audio signal is received from at least one tested person.

In this step, the tested person's breathing or vocalizing audio (e.g., speaking text) can be recorded using the microphone of a mobile device (e.g., a smartphone or a tablet computer). The audio format can be a waveform audio file format or a lossless compression format to guarantee the qualities of subsequently processing audio and extracting audio features. At the same time, the personal information of the “tested person” can be inquired and obtained on any device interface, such as sex, age, height, weight, whether to smoke, etc.

In Step S120, the raw audio signal is processed and noise filtering is performed on the raw audio signal to generate a filtered audio signal.

In this step, the following operations are performed on the raw audio signal.
1. Voice Activity Detection: The long-time voice-free segment or background noise is excluded; and/or
2. Spectrum Subtraction: The voice-free segments around the tested person are used as background noise samples and their average spectrum is calculated. The spectrum is correspondingly subtracted from the entire audio to obtain a clearer voice signal.

After the foregoing process, a “filtered audio signal” is obtained and its signal-to-noise ratio should be significantly greater than that of the raw audio signal. The filtered audio signal is conveniently extracted in the subsequent process.

In Step S130, an audio feature is extracted from the filtered audio signal.

In this step, short-time Fourier transform may be performed on the filtered audio signal to obtain spectrum information. Then, at least one of the following features may be calculated:
1. Spectrum features include Mel-frequency cepstral coefficients (MFCC), delta-MFCC, the distribution of spectrum energy, etc.
2. Vocalizing features include changes in vocal cord vibration, fundamental frequency (FO), jitter, amplitude, harmonic to noise ratio (HNR), etc.

Normalization (e.g., Z-score) may be performed on the initial features to ensure that the dimensions of physical quantities of the audio features of different tested persons are equal.

In Step S140, the audio feature is input into a machine learning model to generate a first predicted pulmonary function parameter.

In this step, a pretrained machine learning model can be established, such as a deep neural network (DNN), a recurrent neural network (RNN), a long short-term memory (LSTM), or a convolutional neural network (CNN) with an attention mechanism, etc. The input layer of the machine learning model includes audio features or their vector values extracted in Step S130. The output layer of the machine learning model generates the “first predicted pulmonary function parameter” that is implemented with a floating-point number or a vector value representing pulmonary function indicators of the tested person, such as a peak expiratory flow, the best peak expiratory flow, an asthma control test score, a forced expiratory volume in one second, and a forced vital capacity.

Furthermore, the machine learning model mainly depends on audio signals, the machine learning model necessarily depends on the tested person's sex, age, or height. As a result, the “first predicted pulmonary function parameter” is often a more generalized or estimated value.

In Step S150, the first predicted pulmonary function parameter is normalized to generate a second predicted pulmonary function parameter.

In this step, the first predicted pulmonary function parameter is corrected based on a preset reference standard to obtain a second predicted pulmonary function parameter that more accurately reflects the individual's physiological condition. The step is specifically described as follows:

### 1. Defining Reference Standard:

The reference standard can be derived from a clinically established formula, such as the “best peak expiratory flow (PEF BEST)” formula or the “forced expiratory volume in one second (FEV1)” formula. These formulas may be obtained from an internal database. Examples include:

$$PEF\_BEST ( male ) = f ( Age , Height ) ,$$

$$PEF\_BEST ( female ) = g ( Age , Height ) ,$$

Alternatively, the formula may be obtained from established medical guidelines, such as the Global Initiative for Asthma (GINA) or local respiratory societies.

### 2. Calculating Ideal Pulmonary Function Value Corresponding to Sex/Age/Height:

For example, if the first predicted pulmonary function parameter is a peak expiratory flow (PEF), the PEF BEST is firstly calculated based on the parameters (Age, Height, Sex) inputted by the tested person.

For example, when the tested person is 40 years old, 170 cm tall, and male, the database provides PEF_BEST (40, 170, male)=600 L/min.

### 3. Normalization Methods:

The normalization methods can include ratio-based correction, difference-based correction, or other linear/non-linear mappings.

### (1) Ratio-Based Correction:

Let P1 be the first predicted pulmonary function parameter and B be the ideal standard.

$$The second predicted pulmonary function parameter = P 1 \times \frac{B}{\tilde{B}} .$$

\tilde{B} is the reference average value used in the machine learning model. This method ensures that the second predicted pulmonary function parameter is generated based on the age/height/sex of the tested person.

### (2) Difference-Based Correction:

Second predicted pulmonary function parameter=P1+α(B-B̅).

α is a weighting constant, and B̅ represents the average standard of either the population for the same sex and the same age or the machine learning model (e.g., the average standard of the labeled audio sample set). This method tunes the first predicted value to be closer to B.

4. Generating Second Predicted Pulmonary Function Parameter: Finally, the normalized (individualized) predicted parameter of the tested person is obtained to correspond to the pulmonary function condition based on age, height, and sex. If PEF=500 L/min is calculated based on the first predicted pulmonary function parameter, the standard for the age/height/sex may be 600 L/min. After normalization (using ratio-based or difference-based correction), the second predicted pulmonary function parameter may be approximately 530˜550 L/min, ensuring the result better satisfies the individual's physiological conditions.

When the embodiment is combined with other embodiments described later, a real pulmonary function parameter (as the labeled audio sample) can be collected to continuously optimize the normalization formula or to update the correction weights. This ensures that age/height/sex accurately affects the pulmonary function parameter.

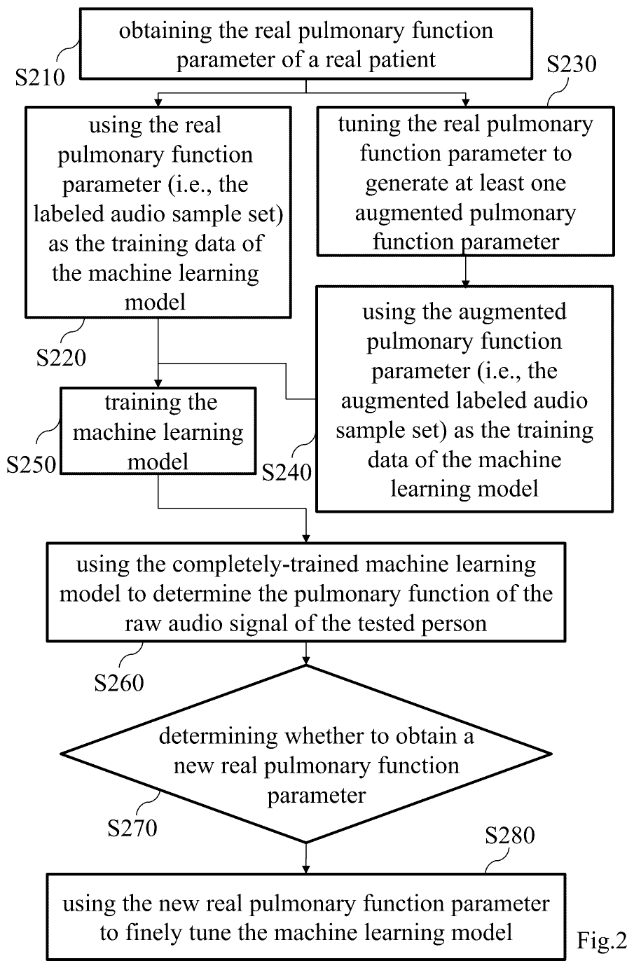

Refer FIG. 2. FIG. 2 is a flowchart of a method for detecting pulmonary function abnormalities based on audio signals according to a second embodiment of the present invention. The flowchart includes Steps S210˜S280.

In Step S210, the real pulmonary function parameter of a real patient is obtained.

In this step, the real pulmonary function parameter of the real patient can be collected using a peak expiratory flow meter (measuring PEF), a spirometer (measuring FEV1, FVC), and asthma control test (ACT) score clinically assessed by a physician. Thus, the real pulmonary function parameter includes, but is not limited to, patient identification (ID), asthma control, control level, sex, age, height, weight, smoking, a peak expiratory flow (PEF), the best peak expiratory flow (PEF BEST), an asthma control test (ACT) score, fractional exhaled nitric oxide (FeNO), a forced expiratory volume in one second (FEV1), a forced vital capacity (FVC), or a combination of these.

The asthma control classification that includes a well-controlled type, a partially controlled type, and an uncontrolled type can be determined based on the guidelines of the GINA or the clinical experiment of physicians.

For adults, the formulas associated with PEF BEST may include:
Male: PEF BEST=(3.8856×Height)−(2.9508×Age)+43.5846
Female: PEF BEST=(4.1028×Height)−(1.611×Age)−173.5476
Example: For Patient A (Male, Age=37, Height=163 cm), the PEF

BEST is 568 L/min. By comparing this to the real PEF=440 L/min, the severity of the condition can be assessed.

Furthermore, the real pulmonary function parameters of 30 patients (as shown in Table 1) serve as a crucial foundation for generating augmented pulmonary function parameters and training the machine learning model in the subsequent process. The details will be described in subsequent embodiments.

TABLE 1
real pulmonary function parameters of 30 patients

| | | | | | | | | | PEF | | | | |
|---|---|---|---|---|---|---|---|---|---|---|---|---|---|
| | asthma | control | | | Height | Weight | | PEF | BEST | | FeNO | FEV1 | FVC |
| ID | control | level | Sex* | Age | (cm) | (kg) | Smoking | (L/min) | (L/min) | ACT | (ppb) | (L) | (L) |
| 1 | well | 1 | 2 | 45 | 158 | 62 | null | 440 | 450 | 22 | 10 | 114% | 116% |
| | controlled | | | | | | | | | | | | |
| 2 | well | 1 | 2 | 36 | 163.5 | 86.2 | null | 550 | 550 | 24 | 17 | N | N |
| | controlled | | | | | | | | | | | | |
| 3 | well | 1 | 2 | 65 | 160 | 67 | null | 200 | 385 | 22 | 12 | 37% | 40% |
| | controlled | | | | | | | | | | | | |
| 4 | partial | 2 | 2 | 65 | 155 | 55 | null | 300 | 351 | 18 | 19 | 88% | 92% |
| | controlled | | | | | | | | | | | | |
| 5 | well | 1 | 2 | 59 | 160.5 | 47.9 | null | 350 | 389 | 21 | 30 | 88% | 100% |
| | controlled | | | | | | | | | | | | |
| 6 | well | 1 | 2 | 65 | 156.5 | 55 | null | 300 | 363 | 22 | 42 | 112% | 115% |
| | controlled | | | | | | | | | | | | |
| 7 | well | 1 | 2 | 63 | 156 | 53 | null | 400 | 400 | 23 | 14 | 132% | 131% |
| | controlled | | | | | | | | | | | | |
| 8 | well | 1 | 2 | 66 | 152 | 47 | null | 300 | 345 | 22 | 21 | 91% | 85% |
| | controlled | | | | | | | | | | | | |
| 9 | well | 1 | 2 | 61 | 168 | 73 | null | 480 | 500 | 19 | 17 | 101% | 103% |
| | controlled | | | | | | | | | | | | |
| 10 | well | 1 | 1 | 70 | 161.5 | 60.9 | null | 560 | 560 | 22 | 51 | 113% | 106% |
| | controlled | | | | | | | | | | | | |
| 11 | partial | 2 | 2 | 83 | 150 | 89.5 | null | 250 | 376 | 21 | 65 | 63% | 68% |
| | controlled | | | | | | | | | | | | |
| 12 | well | 1 | 2 | 22 | 157 | 52 | null | 250 | 589 | 22 | 10 | 65% | 64% |
| | controlled | | | | | | | | | | | | |
| 13 | well | 1 | 1 | 42 | 165 | 70 | null | 550 | 640 | 23 | N | 104% | 104% |
| | controlled | | | | | | | | | | | | |
| 14 | well | 1 | 1 | 63 | 176 | 78 | null | 580 | 580 | 23 | 18 | 124% | 116% |
| | controlled | | | | | | | | | | | | |
| 15 | well | 1 | 1 | 73 | 163 | 67 | Y | 480 | 480 | 23 | 32 | N | N |
| | controlled | | | | | | | | | | | | |
| 16 | partial | 2 | 2 | 36 | 148.5 | 49.3 | null | 330 | 372 | 17 | N | 85% | 82% |
| | controlled | | | | | | | | | | | | |
| 17 | well | 1 | 1 | 67 | 169 | 87 | null | 450 | 503 | 21 | N | 87% | 82% |
| | controlled | | | | | | | | | | | | |
| 18 | well | 1 | 2 | 50 | 168 | 51.5 | null | 350 | 431 | 23 | 39 | N | N |
| | controlled | | | | | | | | | | | | |
| 19 | well | 1 | 2 | 51 | 155 | 86 | null | 300 | 364 | 21 | 5 | 69% | 65% |
| | controlled | | | | | | | | | | | | |
| 20 | well | 1 | 2 | 73 | 160 | 64 | null | 310 | 355 | 22 | 6 | 73% | 76% |
| | controlled | | | | | | | | | | | | |
| 21 | partial | 1 | 1 | 37 | 163 | 72 | null | 440 | 568 | 22 | N | 83% | 85% |
| | controlled | | | | | | | | | | | | |
| 22 | partial | 2 | 1 | 22 | 184 | 80 | null | 600 | 694 | 20 | 108 | 107% | 105% |
| | controlled | | | | | | | | | | | | |
| 23 | well | 1 | 2 | 35 | 163 | 54 | null | 120 | 439 | 21 | 32 | 89% | 91% |
| | controlled | | | | | | | | | | | | |
| 24 | uncontrolled | 3 | 2 | 61 | 158 | 64 | null | 300 | 376 | 21 | N | 75% | 94% |
| 25 | uncontrolled | 1 | 1 | 57 | 175 | 105 | null | 630 | 680 | 22 | N | N | N |
| 26 | uncontrolled | 1 | 1 | 75 | 147.5 | 50.4 | null | 250 | 314 | 20 | N | N | N |
| 27 | well | 1 | 2 | 75 | 170 | 62 | null | 390 | 502 | 23 | 33 | N | N |
| | controlled | | | | | | | | | | | | |
| 28 | well | 1 | 1 | 76 | 160 | 76 | null | 270 | 358 | 23 | 16 | N | N |
| | controlled | | | | | | | | | | | | |
| 29 | well | 1 | 1 | 41 | 155 | 53 | null | 450 | 450 | 23 | 19 | 122% | 119% |
| | controlled | | | | | | | | | | | | |
| 30 | well | 1 | 1 | 40 | 159 | 60 | null | 450 | 460 | 22 | 18 | 93% | 86% |
| | controlled | | | | | | | | | | | | |

*1 = male; 2 = female

In Step S220, the real pulmonary function parameter (i.e., the labeled audio sample set) is used as the training data of the machine learning model.

As shown in Table 1, the real pulmonary function parameter of each patient (ID 1˜30) can be viewed as the labeled audio sample set. The sample set can be divided into a training set, a validation set, or a test set and used for a cross-validation process during model training. However, in real-world scenarios, the pulmonary function parameters may not always be completely recorded, as shown in Table 1. Some real pulmonary function parameters may be missing. Since clinical data are highly valuable, even incomplete patient data can still be used for training the machine learning model.

Even when some real pulmonary function parameters are missing, a pretrained machine learning model can be used to provide the missing data. The detailed process can be completed using the data augmentation of Step S230 described later.

In Step S230, the real pulmonary function parameter is tuned to generate at least one augmented pulmonary function parameter or to provide the missing real pulmonary function parameter in the clinical data of the real patient.

Each patient record in Table 1 represents a specific combination of attributes (e.g., age, height, PEF, etc.). If a certain type (e.g., “partial controlled” patients) has insufficient samples, some parameters are finely tuned to generate virtual patient data, thereby enriching the training data.

### 1. Finely Tuning Method

### (1) Tuning Based on Age/Height/Weight:

For example, consider Patient ID 21 (Male, Age=37, Height=163 cm, Partial Controlled, PEF BEST=568 L/min, PEF=440 L/min). Possible virtual patient data include ID 21a (Age=40, Height=165.3 cm) and ID 21b (Age=35, Height=161.5 cm). In these cases, PEF BEST remains 568 L/min (or an approximate value). Consider ID 21c (Age=45, Height=169.1 cm). PEF BEST remains approximately 568 L/min. If the recalculated PEF BEST, FEV1, or other indicators fall within a reasonable range, the tuned value can be viewed as an acceptable augmented pulmonary function parameter.

### (2) Tuning Based on Asthma Control (ACT) Classification:

If a patient is classified as “uncontrolled”, but the clinical threshold for “uncontrolled” is only slightly different from the threshold for “partial controlled” by the ACT score of 1 point or the FEV1 value of 10%, the ACT score or FEV1 value can be correspondingly tuned to augment one piece of “partial controlled” data.

For example, Patient ID 4 is female, her ACT classification is “partial controlled”, and her ACT score=18. The ACT score of ID 4a is equal to 16. The ACT score of ID 4b is equal to 17. The ACT score of ID 4c is equal to 19. Three virtual patient records are augmented (i.e., 4a, 4b, 4c). Patient ID 16 is female, her ACT classification is “partial controlled”, and her ACT score=17. The ACT score of ID 16a is equal to 16. The ACT score of ID 16b is equal to 18. The ACT score of ID 16c is equal to 19. Three virtual patient records are augmented (i.e., 16a, 16b, 16c). The augmented pulmonary function parameters are shown in Table 2.

TABLE 2
a first part of augmented pulmonary function parameters of virtual patients

| | asthma | control | | | | | | | PEF | | | | |
|---|---|---|---|---|---|---|---|---|---|---|---|---|---|
| ID | control | level | Sex | Age | Height | Weight | Smoking | PEF | BEST | ACT | FeNO | FEV1 | FVC |
| 4 | partial | 2 | 2 | 65 | 155 | 55 | null | 300 | 351 | 18 | 19 | 88% | 92% |
| | controlled | | | | | | | | | | | | |
| 4a | partial | 2 | 2 | 65 | 155 | 55 | null | 300 | 351 | 16 | 19 | 88% | 92% |
| | controlled | | | | | | | | | | | | |
| 4b | partial | 2 | 2 | 65 | 155 | 55 | null | 300 | 351 | 17 | 19 | 88% | 92% |
| | controlled | | | | | | | | | | | | |
| 4c | partial | 2 | 2 | 65 | 155 | 55 | null | 300 | 351 | 19 | 19 | 88% | 92% |
| | controlled | | | | | | | | | | | | |
| 16 | partial | 2 | 2 | 36 | 148.5 | 49.3 | null | 330 | 372 | 17 | N | 85% | 82% |
| | controlled | | | | | | | | | | | | |
| 16a | partial | 2 | 2 | 36 | 148.5 | 49.3 | null | 330 | 372 | 16 | N | 85% | 82% |
| | controlled | | | | | | | | | | | | |
| 16b | partial | 2 | 2 | 36 | 148.5 | 49.3 | null | 330 | 372 | 18 | N | 85% | 82% |
| | controlled | | | | | | | | | | | | |
| 16c | partial | 2 | 2 | 36 | 148.5 | 49.3 | null | 330 | 372 | 19 | N | 85% | 82% |
| | controlled | | | | | | | | | | | | |

### (3) Tuning Based on PEF Values

The PEF value represents the real data blown by the patient, where PEF BEST is the best predicted value calculated based on a formula. For adult patients (male and female), their PEF BEST values can be derived from the established clinical formula based on their sex, age, and height. For pediatric patients (male and female), their PEF BEST values can be derived from the specialized pediatric formula based on their age, height, and weight. The age and height of adult patients are adjusted but their PEF 10 BEST values remain unchanged. The age, height, and weight of pediatric patients are adjusted but their PEF BEST values remain unchanged. For example, Patient ID 21 is male, his ACT classification is “partial controlled”, his age=37, his height=163 cm, and his PEF BEST=568 L/min. By applying the following formulas, different ages (e.g., 40, 35, 45, 50, and 30 years) are randomly assigned to calculate corresponding heights. Let PB represent the PEF BEST value for an adult male, H represent height, and A represent age. The formula is described as follows:

$$PB = ( 3 \text{.8856} * H ) - ( 2 \text{.9508} * A ) + 43 \text{.5846}$$

$$H = [ PB + ( 2.9508 * A ) - 43.58 \; 46 ] / 3.8856$$

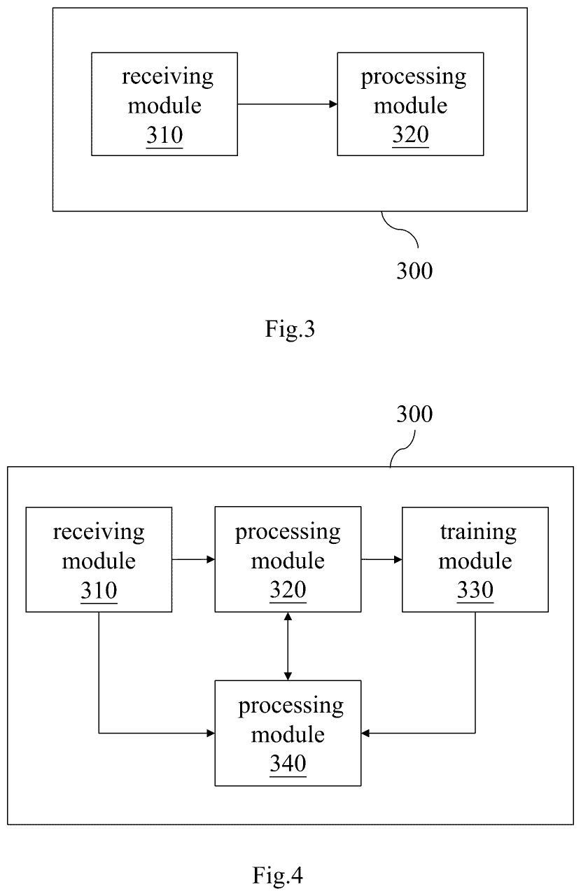

Accordingly, five virtual patient records (i.e., ID 21a, ID 21b, ID 21c, ID 21d, and ID 21e) are augmented. These records include “ID21a, Age 40, Height 165.3”; “ID21b, Age 35, Height 161.5”; “ID21c, Age 45, Height 169.1”; “ID21d, Age 50, Height 172.9”; and “ID21e, Age 30, Height 157.7”. Please refer to FIG. 3. All records retain the same PEF BEST value of 568 as ID 21.

TABLE 3
a second part of augmented pulmonary function parameters of virtual patients

| | asthma | control | | | | | | | PEF | | | | |
|---|---|---|---|---|---|---|---|---|---|---|---|---|---|
| ID | control | level | Sex | Age | Height | Weight | Smoking | PEF | BEST | ACT | FeNO | FEV1 | FVC |
| 21 | partial | 2 | 1 | 37 | 163 | 72 | null | 440 | 568 | 22 | N | 83% | 85% |
| | controlled | | | | | | | | | | | | |
| 21a | partial | 2 | 1 | 40 | 165.3 | 72 | null | 440 | 568 | 22 | N | 83% | 85% |
| | controlled | | | | | | | | | | | | |
| 21b | partial | 2 | 1 | 35 | 161.5 | 72 | null | 440 | 568 | 22 | N | 83% | 85% |
| | controlled | | | | | | | | | | | | |
| 21c | partial | 2 | 1 | 45 | 169.1 | 72 | null | 440 | 568 | 22 | N | 83% | 85% |
| | controlled | | | | | | | | | | | | |
| 21d | partial | 2 | 1 | 50 | 172.9 | 72 | null | 440 | 568 | 22 | N | 83% | 85% |
| | controlled | | | | | | | | | | | | |
| 21e | partial | 2 | 1 | 30 | 157.7 | 72 | null | 440 | 568 | 22 | N | 83% | 85% |
| | controlled | | | | | | | | | | | | |

### 3. Validation of Clinical Reasonability for Tuned Data

### (1) Determination Based on PEF BEST Formula or Range of ACT Score (e.g., ID 16-19):

If the augmented data have Age=50 and Height=172.9, the calculated PEF BEST=568±5 L/min and PEF and FEV1 are correspondingly corrected. If the final difference does not exceed ±10% compared to the clinical data of the original patient, the virtual data are acceptable. If the final difference exceeds ±10% compared to the clinical data of the original patient, the virtual data are excluded.

As Patient ID=24 (Female, Age=61, Height=158, uncontrolled, PEF BEST=376, PEF=300), data “Age-55, 159.5 cm (24b), Age=51, 154 cm (24e)” are augmented. As long as the PEF BEST calculated by the formula is approximately equal to 376, the corresponding the augmented pulmonary function parameter is acceptable.

The different heights of 155.5, 159.5, 150.4, 161, and 154 cm are randomly assigned to Patient ID 24 based on the following formulas, thereby augmenting virtual patients of different ages.

$$Audlt Female PB = ( 4 \text{.1028} * H ) - ( 1.611 * A ) - 173 \text{.5476}$$

$$Audlt Female A = [ ( 4 \text{.1028} * H ) - 173.5 \; 476 - PB ] / 1.611$$

Thus, five pieces of data of the virtual patients (i.e., 24a, 24b, 24c, 24d, and 24e) are augmented in Table 4. They all maintain the original asthma control classification but feature different ages and heights. The data of the virtual patients still meet the reasonable range of the PEF BEST.

TABLE 4
a third part of augmented pulmonary function parameters of virtual patients

| | asthma | control | | | | | | | PEF | | | | |
|---|---|---|---|---|---|---|---|---|---|---|---|---|---|
| ID | control | level | Sex | Age | Height | Weight | Smoking | PEF | BEST | ACT | FeNO | FEV1 | FVC |
| 24 | uncontrolled | 3 | 2 | 61 | 158 | 64 | null | 300 | 376 | 21 | N | 75% | 94% |
| 24a | uncontrolled | 3 | 2 | 55 | 155.5 | 64 | null | 300 | 376 | 21 | N | 75% | 94% |
| 24b | uncontrolled | 3 | 2 | 65 | 159.5 | 64 | null | 300 | 376 | 21 | N | 75% | 94% |
| 24c | uncontrolled | 3 | 2 | 42 | 150.4 | 64 | null | 300 | 376 | 21 | N | 75% | 94% |
| 24d | uncontrolled | 3 | 2 | 69 | 161 | 64 | null | 300 | 376 | 21 | N | 75% | 94% |
| 24e | uncontrolled | 3 | 2 | 51 | 154 | 64 | null | 300 | 376 | 21 | N | 75% | 94% |

In selecting data for augmentation, an index array can also be applied. Firstly, the index array includes 15 elements and the corresponding fields such as ID, Asthma Control, Sex, PEF, PEF BEST, ACT, FEV1, etc. Some fields (e.g., ID, Sex) are fixed with a value of 1, while adjustable parameters such as PEF, PEF BEST, ACT, FEV1, FVC can be set to 0 (disabled) or 1 (enabled).

Based on clinical experience, certain parameters (e.g., FeNO, Smoking) have a relatively rare quantity or include excessive noise in clinical data collection. Thus, they may be set to 0 to avoid negatively affecting augmentation quality. Conversely, if PEF or PEF BEST is sufficient to support the augmentation logic, the parameters may be set to 1. During the “pulmonary function parameter augmentation” process, instead of randomly adjusting Age and Height, the corresponding formulas or ranges are applied to limit Age and Height based on parameters set to 1 in the index array. Examples include:

### 1. PEF Best Formula:

If both PEF and PEF BEST are set to 1 (i.e., enabled), then after adjusting Age and Height, it is determined whether the new combination is still used to obtain a reasonable PEF BEST based on the formula. Additionally, it is determined what percentage the real PEF is of the estimated value.

### 2. ACT or FEV1/FVC

If either ACT=1 or FEV1 and FVC=1, then during the “Uncontrolled→Partial Controlled” transition, it is determined that ACT remains within the range of 16-19 and determined whether the ratio of FEV1 to FVC falls within the clinical classification range.

### Process of Integrating Index Array

### 1. Reading Combination of Index Array:

Example: Index Array=[ . . . , PEF=1, PEF BEST=1, ACT=1, FEV1=0, FVC=0, . . . ].

### 2. Determining Augmentation Basis:

In the index array, when generating the augmented pulmonary function parameter of the virtual patient, it is determined whether the PEF BEST formula and ACT score meet the specific range. However, FEV1 or FVC is not checked. If the adjusted Age and Height are incorporated into the PEF BEST formula to lead to a result that has an error range greater than ±10%, the augmented pulmonary function parameter is excluded. If ACT is also enabled, it is determined whether the ACT scores comply with clinical categorization.

### 3. Generating Valid Augmented Pulmonary Function Parameters:

If the generated data meets clinical conditions, the data “(Age′, Height′, PEF′, PEF_BEST′, ACT′)” is labeled as new virtual patient data that cooperate with the other original values (e.g., fixed fields including Sex, Smoking, etc.) to form a complete augmented labeled audio sample set.

4. Incorporating into Model Training (applied to Step S240):

During training, only parameters set to 1 in the index array are considered for reading features/target fields. If PEF=0 or FEV1=0, the errors of the parameters are not inputted and compared.

In conclusion, each modification to the index array changes the augmented pulmonary function parameters, allowing the machine learning model to iteratively experiment and find the optimal parameter combination. This ensures that the generated virtual patients maintain clinical relevance and effectively improve the data amount of rare classification (e.g., uncontrolled asthma classification) or specific age groups. However, if over-enabling parameters leads to excessive constraints and insufficient augmented data, some parameters can be disabled to relax conditions. Conversely, if too few parameters are enabled, the augmented pulmonary function parameter easily deviates from real clinical conditions. As a result, more parameters can be enabled. Thus, the index array in data augmentation does not directly control how Age, Height, or Weight are adjusted. Instead, it determines which clinical formulas (e.g., PEF BEST, ACT) or classification rules should be referenced during an augmented validation stage and which features should be used in training the machine learning model. This way, specific requirements (e.g., supplementing the number of samples in specific classification) can be augmented while ensuring that the final generated virtual patient data remain clinically reasonable.

In Step S240, the augmented pulmonary function parameter (i.e., the augmented labeled audio sample set) is used as the training data of the machine learning model.

This step inputs the real pulmonary function parameter (i.e., the labeled audio sample set) and the newly generated augmented pulmonary function parameter (i.e., the augmented labeled audio sample set) into a machine learning model (e.g., DNN, CNN, LSTM, etc.) to improve the problem with the scarcity of training data or insufficient samples for specific classification. For example, if the number of uncontrolled classification is too less, the augmented pulmonary function parameters of virtual patients such as ID 21a-21e, ID 16a-16c, . . . can supplement the specific real pulmonary function parameters.

In Step S250, the machine learning model is trained.

During training, the machine learning model can combine the labeled audio sample set (i.e., real patient data) and the augmented labeled audio sample set (i.e., virtual patient data). Since each sample has a corresponding real pulmonary function parameter that is measured, the machine learning model performs prediction on each sample and calculates a difference (i.e., a loss value such as a mean squared error) between a predicted output and a target value. For the loss value, gradient backpropagation is used to dynamically adjust the weights and biases of the machine learning model. This process can further optimize the machine learning model's abilities to more accurately learn various features (e.g., audio features, PEF, FEV1, ACT classification) and improve the accuracy of predicting pulmonary function parameters. To objectively evaluate model performance, a cross-validation process and a trichotomy method are applied in training to ensure that the machine learning model has sufficient accuracy and generalization abilities when analyzing the raw audio signals of unknown tested persons.

Various validation strategies, such as the cross-validation process and the trichotomy method, can be adopted to objectively evaluate the enhanced machine learning model.

In the training strategy of the trichotomy method, the real pulmonary function parameter is used for the training set, the validation set, and the test set because the real pulmonary function parameter is obtained based on clinical measurements or physician's diagnosis. Thus, different conditions of real patients (e.g., different ages, sex, and disease classifications, etc.) can be represented and the real pulmonary function parameter is used as a standard for evaluating the performance of the model in real clinical scenarios. As a result, assigning some real pulmonary function parameters to the test set may really reflect the model's performance on previously unseen real clinical distributions. However, it is recommended that augmented pulmonary function parameters are used only for the training set and the validation set rather than the test set. The augmented pulmonary function parameters are used to generate virtual patient data based on clinical formulas (e.g., PEF BEST) or minor adjustments (e.g., Age, Height). Although these parameters help compensate for scarce samples during training, they still contain synthetic components. If such synthetic data are assigned to the test set, the test results may have overestimation or biases. Thus, the test results do not really represent the model's performance in real clinical scenarios. After testing, the augmented pulmonary function parameters in the training set can significantly compensate for insufficient samples in certain classification (e.g., partial controlled/uncontrolled classification) or specific age groups, allowing the model to learn various features from the diverse samples. In the validation set, the model's behavior in a mixed distribution of synthetic and real data can be monitored in real time. Some augmented data can be put into the validation set to observe how well the model fits the rare classification, thereby continuously adjusting weights and hyperparameters.

Therefore, it should be ensured that the test set only represents real clinical distributions. Thus, it is recommended that the test set entirely or predominantly comes from the real pulmonary function parameters of real clinical patients to maintain the determination accuracy of the machine learning model.

In Step S260, the completely trained machine learning model is used to determine the pulmonary function of the raw audio signal of the tested person.

This step performs Steps S110˜S150 in FIG. 1, uses the initial training of the completely trained machine learning model in Steps S210˜S240, and directly inputs the raw audio signal (after preprocessing and feature extraction) of the unknown tested person into the machine learning model. Based on the learned weights, the machine learning model outputs corresponding pulmonary function parameters such as PEF, FEV1, ACT, or other clinical indicators.

Furthermore, the machine learning model can reference the normalization standard (e.g., PEF BEST calculated based on age and height) or asthma control classification to provide the tested person with results such as “green light (80%˜100%)”, “yellow light (60%˜80%)” or “red light (<60%)”. Please see Table 5 below for the specific light signals corresponding to PEF BEST. If the results show possible moderate or severe abnormalities, the tested person may be advised to go to a medical institution for further examination or treatment.

TABLE 5
Correspondence Table of Severity of Peak Expiratory Flow (PEF)

| | Best estimation | Green light | Yellow light | Red light |
|---|---|---|---|---|
| | value | 80%~100% | 60%~80% | <60% |
| | (PEF BEST) | (PEF) | (PEF) | (PEF) |
| Adult | 524 | 419~524 | 314~419 | <314 |
| (male) | | | | |
| Adult | −174 | −139~−174 | −104~−139 | \|PEF\| < \|−104\| |
| (female) | | | | |
| child | −131 | −104~−131 | −78~−104 | \|PEF\| < \|−78\| |
| (male) | | | | |
| child | −99 | −79~−99 | −59~−79 | \|PEF\| < \|−59\| |
| (female) | | | | |

In Step S270, it is determined whether a new real pulmonary function parameter is obtained.

In this step, new real pulmonary function parameters can be examined at regular check-ups (e.g., weekly or monthly). Alternatively, new real pulmonary function parameters can be obtained after specific events (e.g., when a patient returns for an official pulmonary function test). Take some patients as examples. After a physician evaluates Patient ID 30 using a spirometer, blood oxygen analysis, or ACT (Asthma Control Test), new real pulmonary function measurement values (e.g., PEF, FEV1, ACT) may be obtained. Alternatively, new patient data may be provided, such as Patient ID 31. If no new real pulmonary function parameters are available, the process returns to Step S260 and maintains the currently trained machine learning model while continuing to provide results for determining the audio signals of the unknown tested persons.

In Step S280, the machine learning model is finely tuned using the new real pulmonary function parameter.

If Step S270 determines that new real patient data are generated, the new real patient data are combined with the corresponding real pulmonary function parameters to form an incremental labeled data set. Additionally, following the operation logic of Steps S230 to S240, age or height is adjusted to generate a new augmented pulmonary function parameter based on clinical formulas (e.g., PEF BEST, ACT classification) to supplement the incremental labeled data set.

In general, when new training data (i.e., new real pulmonary function parameters) are generated, the machine learning model typically adopts either full retraining or naïve incremental training. Full retraining and naïve incremental training respectively have apparent drawbacks. Full retraining significantly increases training time and resource costs and needs to refit all parameters. In medical scenarios, it may not be practical for real-time clinical applications (e.g., outpatient clinics). Naive incremental training trains the model only with new data and leads to catastrophic forgetting phenomena. In the phenomena, the model overfits new data while forgetting the learned knowledge of old data. Finally, the model reduces its prediction abilities on earlier distributions.

After testing, the embodiment adopts a fine-tuning retraining mechanism with partial layer freezing. The retraining mechanism retains most of the weights of the old model and performs open-ended training on only the last few layers or specific subnetworks. In addition, the model observes both some old data and newly added data to update the weights. If the feature distribution of new and old data differs significantly when using the training method, fine-tuning only the last few layers may not be sufficient to learn new sample features. A trade-off must be made between the number of frozen layers and learnable parameters. However, in medical applications, data variability is relatively low (as it focuses on specific clinical values). The amount of new data that is newly added every time is usually small. If a small amount of new data is newly added every time, full retraining is performed such that the time and resource costs will be too huge and clinically impractical. Fine-tuning allows to update only some weights or specified blocks, significantly shortening the cyclic training time. Even if the feature distribution of new data in the future is significantly different from the old feature distribution, alternative incremental learning strategies (such as replay-based mechanisms) can be considered. However, fine-tuning training remains the most efficient retraining solution in the embodiment.

Please refer to FIG. 3. FIG. 3 is a diagram schematically illustrating a system for detecting pulmonary function abnormalities based on audio signals according to a first embodiment of the present invention.

According to an embodiment, as shown in FIG. 3, a system for detecting pulmonary function abnormalities based on audio signals (hereafter referred to as “the system 300”) can be implemented in mobile devices, personal computers, servers, or cloud computing platforms. The purpose of the system 300 is to determine the pulmonary function parameters of the breathing and/or vocalizing audio of the tested person, while continuously updating the model using real or augmented pulmonary function parameters.

The system 300 includes a receiving module 310 and a processing module 320. The receiving module 310 is configured to receive or extract the raw audio signal of at least one tested person. The raw audio signal may include the breathing sounds, coughing sounds, or spoken voices of the tested person. For example, audio may be collected by a mobile device or an ear-worn microphone and transmitted to the system 300 via a network or a local channel. The receiving module 310 can also simultaneously extract the patient's basic information such as age, sex, height, etc. for use in subsequent normalization or analysis steps.

The processing module 320 is coupled to the receiving module 310. The processing module 320 is responsible for performing noise filtering and feature extraction on the raw audio signal and then inputting the processed audio signal into the machine learning model to generate a first predicted pulmonary function parameter. Then, based on a preset reference standard (such as PEF BEST, the range of ACT score), the first predicted pulmonary function parameter is converted into a second predicted pulmonary function parameter.

The noise filtering performed by the processing module 320 can be implemented with spectrum subtraction or voice activity detection to reduce background noise or invalid audio segments and generate a filtered audio signal that retains the audio features as much as possible. Feature extraction, for example, uses acoustic features such as Mel-frequency cepstral coefficients (MFCC) or fundamental frequency (FO) jitter, amplitude shimmer, etc. as input to the machine learning model. In terms of prediction and normalization, the processing module 320 makes inferences on the extracted feature vectors to obtain a first predicted pulmonary function parameter (e.g., PEF, FEV1 . . . ), and then adjusts the parameter to a more individual second predicted pulmonary function parameter based on a preset reference standard (age, height, etc.).

Refer to FIG. 4. FIG. 4 is a diagram schematically illustrating a system for detecting pulmonary function abnormalities based on audio signals according to a second embodiment of the present invention.

According to another embodiment, the system 300 further includes a training module 330 coupled to the processing module 320 and configured to obtain the real pulmonary function parameter of at least one patient (such as clinically measured PEF, ACT, FEV1 or FVC). The training module 330 either uses the real pulmonary function parameter as the labeled audio sample set of the machine learning model or uses the real pulmonary function parameter to calibrate or validate the second predicted pulmonary function parameter. The model training process can be implemented with the cross-validation process or the trichotomy method (training set/validation set/test set) to objectively evaluate the model performance and update the weights by calculating the error value. Furthermore, if the parameters of new patients are generated, they can be used as an incremental labeled data set included in the labeled audio sample set and then be retrained or finely tuned to ensure that the model maintains good accuracy in long-term applications.

According to another embodiment, the system 300 further includes a data augmentation module 340 coupled to the receiving module 310, the processing module 320, and the training module 330 and configured to tune the real pulmonary function parameter to generate at least one augmented pulmonary function parameter. This process can adjust age, height or asthma control score, etc. to obtain the physiological conditions of the virtual patient, thereby calculating the augmented pulmonary function parameters that meet the acceptable clinical range and performing the following purposes:
(1) Sample Supplementation: The rare disease classification (such as partially controlled or uncontrolled) is finely tuned to ensure that the machine learning model obtains sufficient training examples in this classification.
(2) Determination of Clinical Reasonability: The PEF BEST formula or the range of asthma control score is used to determine that the augmented samples do not deviate from clinical common sense.
(3) Retraining/Calibration: The processing module 320 can compare the difference between the predicted value and the expected value of the augmented labeled audio sample set, update the parameters of the machine learning model, and finally improve the accuracy of detecting pulmonary function abnormalities.

The embodiments described above are only to exemplify the invention but not to limit the scope of the invention. Therefore, any equivalent modification or variation according to the shapes, structures, features, or spirit disclosed by the invention is to be also included within the scope of the invention.

What is claimed is:

1. A method for detecting pulmonary function abnormalities based on audio signals, comprising:
receiving from at least one tested person a raw audio signal that includes breathing and/or vocalizing audio of the at least one tested person;
processing the raw audio signal and performing noise filtering on the raw audio signal to generate a filtered audio signal;
extracting at least one audio feature from the filtered audio signal, wherein the at least one audio feature includes a spectrum feature and/or a vocalizing feature;
inputting the at least one audio feature into a machine learning model to generate a first predicted pulmonary function parameter; and
normalizing the first predicted pulmonary function parameter and converting the first predicted pulmonary function parameter into a second predicted pulmonary function parameter based on a preset reference standard, wherein the reference standard provides a basis for normalization according to sex, age, and height of the at least one tested person.

2. The method according to claim 1, further comprising a training process that includes:
obtaining a real pulmonary function parameter of at least one patient; and
either using the real pulmonary function parameter as a labeled audio sample set of the machine learning model or using the real pulmonary function parameter to calibrate or validate the second predicted pulmonary function parameter.

3. The method according to claim 2, further comprising:
tuning the real pulmonary function parameter to generate at least one augmented pulmonary function parameter; and
either using the at least one augmented pulmonary function parameter as an augmented labeled audio sample set of the machine learning model or using the at least one augmented pulmonary function parameter to calibrate or validate the second predicted pulmonary function parameter.

4. The method according to claim 3, further comprising:
either calculating a difference value between the labeled audio sample set and the augmented labeled audio sample set or calculating a ratio of the labeled audio sample set to the augmented labeled audio sample set; and
updating a parameter weight of the machine learning model and using the difference value or the ratio as an error value to calibrate predicted accuracy of the machine learning model for pulmonary function abnormalities.

5. The method according to claim 3, further comprising:
performing a cross-validation process to divide the labeled audio sample set and the augmented labeled audio sample set into subsets and using each of the subsets as a validation set in turn while the remaining subsets are used as the training set to evaluate predicted accuracy of the machine learning model for pulmonary function abnormalities; and
selecting the machine learning model with an optimal model configuration based on average accuracy or errors measured by the cross-validation process.

6. The method according to claim 3, further comprising:
dividing the labeled audio sample set into a training set, a validation set, and a test set and dividing the augmented labeled audio sample set into the training set and the validation set;
combining the training sets that are divided from the labeled audio sample set and the augmented labeled audio sample set to train the machine learning model;
tuning hyperparameters or a network structure of the machine learning model based on predicted results of the validation set that are divided from the labeled audio sample set and the augmented labeled audio sample set; and
using the test set divided from the labeled audio sample set to validate predicted accuracy of the machine learning model for pulmonary function abnormalities after tuning the hyperparameters or the network structure.

7. The method according to claim 3, wherein the first predicted pulmonary function parameter, the second predicted pulmonary function parameter, the real pulmonary function parameter, and the augmented pulmonary function parameter include a peak expiratory flow, a best peak expiratory flow, an asthma control test score, a forced expiratory volume in one second, a forced vital capacity, or a combination of these.

8. The method according to claim 3, wherein the step of tuning the real pulmonary function parameter comprises:
correcting at least one of age, height, and weight of the at least one patient to obtain a physiological condition of a virtual patient;
calculating the augmented pulmonary function parameter corresponding to the physiological condition, wherein the augmented pulmonary function parameter is derived by a clinical formula and/or a physiological model; and
determining whether the augmented pulmonary function parameter falls within an acceptable clinical range:
if yes, including the augmented pulmonary function parameter into the augmented labeled audio sample set; and
if no, excluding the augmented pulmonary function parameter.

9. The method according to claim 8, further comprising:
receiving a new real pulmonary function parameter of at least one new patient;
including the new real pulmonary function parameter into an incremental labeled data set and including the incremental labeled data set update the labeled audio sample set; and
finely tuning the machine learning model and using the incremental labeled data set to perform another training on the machine learning model.

10. The method according to claim 1, wherein the noise filtering includes a spectrum subtraction, which selects voice-free segments from the raw audio signal as background noise, calculates an average spectrum of the background noise, and linearly subtracts the average spectrum from a spectrum of the raw audio signal.

11. The method according to claim 1, wherein the noise filtering includes voice activity detection that is determined based on a short-time energy threshold, short-time energy is an average value of square values of amplitudes of the raw audio signal within a predetermined time window, the short-time energy threshold is a dynamic reference value calculated based on environmental background noise, the short-time energy threshold is used to determine a voice activity, and a method for determining the voice activity includes:
comparing the short-time energy with the short-time energy threshold in each time window, and when the short-time energy in consecutive time windows is greater than the short-time energy threshold, determining that the voice activity begins;
when the short-time energy in consecutive time windows is less than the short-time energy threshold, determining that the voice activity ends; and
extracting the voice activity in a time period as a valid voice segment and excluding background noise.

12. A system for detecting pulmonary function abnormalities based on audio signals, comprising:
a receiving module configured to receive or extract from at least one tested person a raw audio signal that includes breathing and/or vocalizing audio of the at least one tested person; and
a processing module coupled to the receiving module and configured to perform noise filtering on the raw audio signal to generate a filtered audio signal, extract at least one audio feature from the filtered audio signal, input the at least one audio feature into a machine learning model to generate a first predicted pulmonary function parameter, and convert the first predicted pulmonary function parameter into a second predicted pulmonary function parameter based on a preset reference standard.

13. The system according to claim 12, further comprising a training module coupled to the processing module and configured to obtain a real pulmonary function parameter of at least one patient, and the training module is configured to either use the real pulmonary function parameter as a labeled audio sample set of the machine learning model or use the real pulmonary function parameter to calibrate or validate the second predicted pulmonary function parameter.

14. The system according to claim 13, further comprising a data augmentation module coupled to the receiving module, the processing module, and the training module and configured to tune the real pulmonary function parameter to generate at least one augmented pulmonary function parameter, and the data augmentation module is configured to either use the at least one augmented pulmonary function parameter as an augmented labeled audio sample set of the machine learning model or use the at least one augmented pulmonary function parameter to calibrate or validate the second predicted pulmonary function parameter.

15. The system according to claim 14, wherein the processing module is configured to either calculate a difference value between the labeled audio sample set and the augmented labeled audio sample set or calculate a ratio of the labeled audio sample set to the augmented labeled audio sample set, update a parameter weight of the machine learning model, and use the difference value or the ratio as an error value to calibrate predicted accuracy of the machine learning model for pulmonary function abnormalities.

16. The system according to claim 14, wherein the processing module is further configured to perform a cross-validation process to divide the labeled audio sample set and the augmented labeled audio sample set into subsets, use each of the subsets as a training set and a validation set in turn to evaluate predicted accuracy of the machine learning model for pulmonary function abnormalities, and select the machine learning model with an optimal model configuration based on average accuracy or errors measured by the cross-validation process.

17. The system according to claim 14, wherein the processing module is further configured to divide the labeled audio sample set into a training set, a validation set, and a test set, divide the augmented labeled audio sample set into the training set and the validation set, combine the training sets that are divided from the labeled audio sample set and the augmented labeled audio sample set to train the machine learning model, and tune hyperparameters or a network structure of the machine learning model based on predicted results of the validation set that are divided from the labeled audio sample set and the augmented labeled audio sample set, and the processing module uses the test set divided from the labeled audio sample set to validate predicted accuracy of the machine learning model for pulmonary function abnormalities after tuning the hyperparameters or the network structure.

18. The system according to claim 14, wherein the first predicted pulmonary function parameter, the second predicted pulmonary function parameter, the real pulmonary function parameter, and the augmented pulmonary function parameter include a peak expiratory flow, a best peak expiratory flow, an asthma control test score, a forced expiratory volume in one second, a forced vital capacity, or a combination of these.

19. The system according to claim 14, wherein when tuning the real pulmonary function parameter, the data augmentation module corrects age, height, or weight of the at least one patient or a combination of these to obtain a physiological condition of a virtual patient, calculates the augmented pulmonary function parameter corresponding to the physiological condition, and determines whether the augmented pulmonary function parameter falls within an acceptable clinical range, the augmented pulmonary function parameter is derived by a clinical formula and/or a physiological model, and when the augmented pulmonary function parameter falls within the acceptable clinical range, the data augmentation module includes the augmented pulmonary function parameter into the augmented labeled audio sample set.

20. The system according to claim 19, wherein the training module is further configured to receive a new real pulmonary function parameter of at least one new patient, include the new real pulmonary function parameter into an incremental labeled data set, include the incremental labeled data set update the labeled audio sample set, finely tune the machine learning model, and use the incremental labeled data set to perform another training on the machine learning model.

21. The system according to claim 12, wherein the noise filtering includes a spectrum subtraction, which selects voice-free segments from the raw audio signal as background noise, calculates an average spectrum of the background noise, and linearly subtracts the average spectrum from a spectrum of the raw audio signal.

22. The system according to claim 12, wherein the noise filtering includes voice activity detection that is determined based on a short-time energy threshold, short-time energy is an average value of square values of amplitudes of the raw audio signal within a predetermined time window, the short-time energy threshold is a dynamic reference value calculated based on environmental background noise, the short-time energy threshold is used to determine a voice activity, and a method for determining the voice activity includes:
comparing the short-time energy with the short-time energy threshold in each time window, and when the short-time energy in consecutive time windows is greater than the short-time energy threshold, determining that the voice activity begins;
when the short-time energy in consecutive time windows is less than the short-time energy threshold, determining that the voice activity ends; and
extracting the voice activity in a time period as a valid voice segment and excluding background noise.
